(19) United States
(12) Patent Application Publication
(10) Pub. No.: US 2007/0066536 A1
(43) Pub. Date: Mar. 22, 2007

(54) Methods for treating hormone associated conditions using a combination of LHRH antagonists and specific estrogen receptor modulators

(75) Inventor: Marc B. Garnick, Brookline, MA (US)

Correspondence Address: LAHIVE & COCKFIELD, LLP, ONE POST OFFICE SQUARE, BOSTON, MA, 02109-2127, US

(73) Assignee: Praecis Pharmaceuticals, Inc., Waltham, MA (US)

(21) Appl. No.: 11/522,038

(22) Filed: Sep. 15, 2006

Related U.S. Application Data
(63) Continuation of application No. 10/619,684, filed on Jul. 14, 2003, now abandoned.
Application No. 10/619,684 is a continuation of application No. PCT/US02/00751, filed on Jan. 9, 2002.
(60) Provisional application No. 60/262,494, filed on Jan. 17, 2001.

(51) Int. Cl. A61K 38/09; A61K 31/56

(52) U.S. Cl. 514/15; 514/171

(57) ABSTRACT

Methods for treating hormone associated conditions, such as endometriosis, uterine leiomata, ovarian cancer, breast cancer, or vaginal bleeding, using LHRH antagonists and selective estrogen receptor modulators are disclosed. The methods include administering to a subject a combination of an LHRH antagonist and a selective estrogen receptor modulator. Pharmaceutical compositions and kits for use in the methods of the invention are also provided.

### RELATED APPLICATIONS

This application is a continuation of U.S. patent application Ser. No. 10/619,684, filed Jul. 14, 2003, which is a continuation of International Application No. PCT/US02/00751, filed Jan. 9, 2002, which claims priority to U.S. Provisional Patent Application No. 60/262,494 filed Jan. 17, 2001, the entire contents of each of which are incorporated herein by reference.

### BACKGROUND OF THE INVENTION

Hormone associated conditions, such as endometriosis, uterine leiomata, ovarian cancer or breast cancer present one of the most serious threats to women's health today.

Endometriosis is the ectopic presence of endometrial type glands and stroma in sites which are outside of the uterus. This ectopic occurrence of endometrial tissue frequently forms cysts containing altered blood. The condition results in debilitating pain for millions of women worldwide and particularly occurs in conjunction with the monthly proliferation of endometrial tissue. Endometriosis is frequently a lifelong condition.

Ovarian cancer is often called the silent killer because many times there are no symptoms until the disease has progressed to an advanced stage. One-third of American women will get some form of cancer in their lifetime and approximately 1.4% of those cases will be cancer involving one or both ovaries.

Dysfunctional (or abnormal) uterine bleeding (DUB) is a problem that often affects women as they start to get periods and as they get closer to menopause, although any woman who menstruates can experience DUB. The main symptoms are prolonged and/or irregular menstrual bleeding. The bleeding may be irregular spotting during the cycle, but sometimes the bleeding is so heavy that a woman can't participate in her normal day-to-day activities, such as work and exercise.

One of the most effective treatments for hormone associated conditions is the administration to an affected subject of an LHRH antagonist. LHRH antagonists inhibit the release of Luteinizing hormone (LH) and follicle-stimulating hormone (FSH) by blocking the action of Luteinizing hormone-releasing hormone (LHRH; also known as gonadotropin-releasing hormone or GnRH).

A typical problem, however, that is frequently encountered with the use of LHRH antagonists is a series of side effects stemming from protracted severe estrogen deprivation. These side effects include hot flashes, bone loss, and increased susceptibility to cardiovascular disease, such as hyperlipidemia, restenosis, hypertension, and thrombosis.

### SUMMARY OF THE INVENTION

The present invention provides methods for treating a hormone associated condition in a subject by using a combination of an LHRH antagonist and a selective estrogen receptor modulator (SERM). The present invention is based, at least in part, on the discovery that the use of an LHRH antagonist in combination with a selective estrogen receptor modulator allows for the treatment of a hormone associated condition while at the same time avoiding the side effects normally associated with the use of LHRH antagonists.

Accordingly, the present invention provides a method for treating (e.g., therapeutically or prophylactically) a hormone associated condition, e.g., endometriosis, ovarian cancer, breast cancer, polycystic ovary syndrome, uterine leiomata, dysfunctional uterine bleeding, premenstrual syndrome, vaginal bleeding, or uterine fibroids, in a subject. The method includes administering to a subject a combination of an LHRH antagonist and a selective estrogen receptor modulator, thereby treating a hormone associated condition in the subject. In one embodiment, the subject is a mammal, preferably a human.

In a preferred embodiment, the LHRH antagonist is a decapeptide or a nonapeptide compound having a D-asparagine, an L-asparagine, a D-glutamine, or an L-glutamine at a position corresponding to position 6 of naturally occurring LHRH, or a pharmaceutically acceptable salt thereof. In one embodiment, the LHRH antagonist is a peptide compound comprising a structure: A-B-C-D-E-F-G-H-I-J, wherein A is pyro-Glu, Ac-D-Nal, Ac-D-Qal, Ac-Sar, or Ac-D-Pal, or an analogue thereof; B is His or 4-Cl-D-Phe, or an analogue thereof; C is Trp, D-Pal, D-Nal, L-Nal-D-Pal(N—O), or D-Trp, or an analogue thereof; D is Ser, or an analogue thereof; E is N-Me-Ala, Tyr, N-Me-Tyr, Ser, Lys(iPr), 4-Cl-Phe, His, Asn, Met, Ala, Arg or Ile, or an analogue thereof; F is D-Asn or D-Gln; G is Leu or Trp, or an analogue thereof; H is Lys(iPr), Gln, Met, or Arg, or an analogue thereof; I is Pro, or an analogue thereof; and J is Gly-NH₂ or D-Ala-NH₂, or an analogue thereof; or a pharmaceutically acceptable salt thereof. In another embodiment, the LHRH antagonist is a peptide compound comprising a structure: A-B-C -D-E-F-G-H-I-J, wherein A is pyro-Glu, Ac-D-Nal, Ac-D-Qal, Ac-Sar, or Ac-D-Pal, or an analogue thereof; B is His or 4-Cl-D-Phe, or an analogue thereof; C is Trp, D-Pal, D-Nal, L-Nal-D-Pal(N—O), or D-Trp, or an analogue thereof; D is Ser, or an analogue thereof; E is N-Me-Ala, Tyr, N-Me-Tyr, Ser, Lys(iPr), 4-Cl-Phe, His, Asn, Met, Ala, Arg or Ile, or an analogue thereof; F is D-Asn; G is Leu or Trp, or an analogue thereof; H is Lys(iPr), Gln, Met, or Arg, or an analogue thereof; I is Pro, or an analogue thereof; and J is Gly-NH₂ or D-Ala-NH₂, or an analogue thereof; or a pharmaceutically acceptable salt thereof.

In preferred embodiments, the LHRH antagonist is a peptide compound comprising a structure: Ac-D-Nal-4-Cl-D-Phe-D-Pal-Ser-N-Me-Tyr-D-Asn-Leu-Lys(iPr)-Pro-D-Ala-NH₂ or Ac-D-Nal-4-Cl-D-Phe-D-Pal-Ser-Tyr-D-Asn-Leu-Lys(iPr)-Pro-D-Ala-NH₂ or a pharmaceutically acceptable salt thereof.

In another preferred embodiment, the LHRH antagonist has an ED₅₀ for histamine release in a standard in vitro histamine release assay of at least 3 μg/ml, 5 μg/ml, or 10 μg/ml.

In one embodiment, the selective estrogen receptor modulator is raloxifene (Evista®) tamoxifen, clomifen, lasofoxifene, idoxifene, droloxifene, levomeloxifene, and toremifine.

In one embodiment, the LHRH antagonist is administered to the subject using a pharmaceutical composition comprising a solid ionic complex of the LHRH antagonist and a carrier macromolecule, wherein the carrier and LHRH antagonist used to form the complex are combined at a weight ratio of carrier:LHRH antagonist of 0.8:1 to 0.1:1. In a preferred embodiment, the complex is not a microcapsule. Ranges intermediate to the above recited values, e.g., 0.8:1 to 0.4:1, 0.6:1 to 0.2:1, or 0.5:1 to 0.1:1 are also intended to be part of this invention. Other possible ratios of carrier: LHRH antagonist include 0.5:1, 0.4:1, 0.3:1, 0.25:1, 0.15:1, and 0.1:1. Moreover, ranges of values using a combination of any of the above recited values as upper and/or lower limits are intended to be included.

In another embodiment, the LHRH antagonist is administered to the subject using a pharmaceutical composition comprising a solid ionic complex of an LHRH antagonist and a carrier macromolecule, wherein the LHRH antagonist content of said complex is at least 40% by weight, preferably at least 45%, 50%, 55%, 57%, 60%, 65%, 70%, 75%, 80%, 85%, 90%, or 95% by weight. Ranges intermediate to the above recited values, e.g., at least about 50% to about 80%, at least about 60% to about 90%, or at least about 57% to about 80%, are also intended to be part of this invention. For example, ranges of values using a combination of any of the above recited values as upper and/or lower limits are intended to be included.

In another embodiment, the dosage of the LHRH antagonist and/or the selective estrogen receptor modulator is about 10-500 mg/month in a sustained-release form, about 20-300 mg/month in a sustained-release form, or about 30-200 mg/month in a sustained-release form. In a preferred embodiment, the dosage of the LHRH antagonist and/or the selective estrogen receptor modulator is about 30-120 mg/month in a sustained-release form. Ranges intermediate to the above recited values, e.g., about 10-200 mg/month in a sustained-release form, about 30-250 mg/month in a sustained-release form, or about 100-200 mg/month in a sustained-release form, are also intended to be part of this invention. For example, ranges of values using a combination of any of the above recited values as upper and/or lower limits are intended to be included. The above recited dosages may also be calculated and expressed in mg/kg/day (based on an average subject weight of about 73 kg). Accordingly, in another embodiment, the dosage of the LHRH antagonist and/or the selective estrogen receptor modulator is about 5-500 μg/kg/day, about 10-400 μg/kg/day, or about 20-200 μg/kg/day. In a preferred embodiment, the dosage of the LHRH antagonist and/or the selective estrogen receptor modulator is about 100 μg/kg/day.

In yet another embodiment, the LHRH antagonist and/or the selective estrogen receptor modulator are administered to the subject orally, intravenously, intramuscularly, or subcutaneously, preferably in a pharmaceutically acceptable formulation. The pharmaceutically acceptable formulation is preferably a lipid-based formulation, a saline based formulation, or a manitol based formulation. The LHRH antagonist and/or the selective estrogen receptor modulator can be administered in the same formulation or in separate formulations. In other preferred embodiments, the LHRH antagonist and/or the selective estrogen receptor modulator are administered simultaneously. In yet other preferred embodiments, the LHRH antagonist and/or the selective estrogen receptor modulator are administered at different times, e.g., the LHRH antagonist may be administered to the subject within 1, 2, 3, 4, 5, 6, 7, 8, 9, 10, 11, or 12 hours after the selective estrogen receptor modulator is administered to the subject or within 1, 2, 3, 4, 5, 6, 7, 8, 9, 10, 11, or 12 hours before the selective estrogen receptor modulator is administered to the subject.

In another aspect, the present invention provides a method for treating endometriosis in a subject by administering to a subject a combination of an LHRH antagonist and a selective estrogen receptor modulator, thereby treating endometriosis in the subject.

In a further subject, the present invention features a method for treating ovarian cancer in a subject by administering to a subject a combination of an LHRH antagonist and a selective estrogen receptor modulator, thereby treating ovarian cancer in the subject.

In yet another aspect, the invention provides a method for treating breast cancer in a subject by administering to a subject a combination of an LHRH antagonist and a selective estrogen receptor modulator, thereby treating breast cancer in the subject.

In another aspect, the present invention provides a method for treating polycystic ovary syndrome in a subject by administering to a subject a combination of an LHRH antagonist and a selective estrogen receptor modulator, thereby treating polycystic ovary syndrome in the subject.

In a further aspect, the present invention provides a method for treating uterine leiomata in a subject by administering to a subject a combination of an LHRH antagonist and a selective estrogen receptor modulator, thereby treating uterine leiomata in the subject.

In another aspect, the present invention features a method for treating dysfunctional uterine bleeding in a subject by administering to a subject a combination of an LHRH antagonist and a selective estrogen receptor modulator, thereby treating dysfunctional uterine bleeding in the subject.

In yet another aspect, the present invention features a method for treating premenstrual syndrome in a subject by administering to a subject a combination of an LHRH antagonist and a selective estrogen receptor modulator, thereby treating premenstrual syndrome in the subject.

In another aspect, the present invention features a method for treating vaginal bleeding in a subject by administering to a subject a combination of an LHRH antagonist and a selective estrogen receptor modulator, thereby treating vaginal bleeding in the subject. In one embodiment, the vaginal bleeding is due to thrombocytopenia, for example, caused by chemotherapy treatment. In another embodiment, the subject is suffering from a proliferative disorder, e.g., acute myeloid leukemia. In another embodiment, the subject is a transplant recipient.

In yet another aspect, the present invention features a method for treating uterine fibroids in a subject by administering to a subject a combination of an LHRH antagonist and a selective estrogen receptor modulator, thereby treating uterine fibroids in the subject.

The present invention also features pharmaceutical compositions containing an LHRH antagonist and a selective estrogen receptor modulator in an amount effective to treat a hormone associated condition in a subject. In one embodiment, the pharmaceutical compositions may further include another drug suitable for treating a hormone associated condition in a subject. In a preferred embodiment, the pharmaceutical composition further includes a pharmaceutically acceptable carrier, e.g., a lipid-based carrier.

In another aspect, the invention features kits including an LHRH antagonist and a selective estrogen receptor modulator in an amount effective to treat a hormone associated condition in a subject. In one embodiment, the kit may further include instructions for use and/or another drug suitable for treating a hormone associated condition in a subject.

Other features and advantages of the invention will be apparent from the following detailed description and claims.

### DETAILED DESCRIPTION OF THE INVENTION

The present invention provides methods for treating a hormone associated condition in a subject by using a combination of an LHRH antagonist and a selective estrogen receptor modulator (SERM). The present invention is based, at least in part, on the discovery that the use of an LHRH antagonist in combination with a selective estrogen receptor modulator allows for the treatment of a hormone associated condition while at the same time avoiding the side effects normally associated with the use of LHRH antagonists.

Accordingly, the present invention provides a method for treating a hormone associated condition, e.g., endometriosis, ovarian cancer, breast cancer, polycystic ovary syndrome, uterine leiomata, dysfunctional uterine bleeding, premenstrual syndrome, vaginal bleeding, or uterine fibroids, in a subject. The method includes administering to a subject a combination of an LHRH antagonist and a selective estrogen receptor modulator, thereby treating a hormone associated condition in the subject.

As used herein, the term “hormone associated condition” includes any disease, disorder, or condition associated with a sex hormone, e.g., estrogen or progesterone. Hormone associated conditions include conditions, diseases, or disorders which affect the organs of the reproductive system, e.g., the uterus or the vagina; conditions, diseases, or disorders which involve an imbalance in the levels of a reproductive-hormone in a subject; and conditions, diseases, or disorders affecting the ability of a subject to reproduce. Examples of hormone associated conditions include endometriosis, vaginal bleeding, infertility, ovarian cancer, uterine fibroids, breast cancer, premenstrual syndrome, polycystic ovary syndrome, ovarian cysts, uterine leiomata, and dysfunctional uterine bleeding.

As used herein, the term “subject” includes warm-blooded animals, preferably mammals, including humans. In a preferred embodiment, the subject is a primate. In an even more preferred embodiment, the primate is a human.

As used herein, the term “LHRH antagonist” includes a compound that inhibits the gonadotropin releasing hormone receptor such that release of gonadotropins is inhibited. The term “LHRH antagonist” may be used interchangeably with the term “LHRH-R antagonist” to include compounds that inhibit LHRH-R such that release of both LH and FSH is inhibited. LHRH antagonists of the present invention are suitable for in vivo administration, e.g., they have good water solubility and/or low histamine-releasing activity. Preferred LHRH antagonists are those having low histamine-releasing activity (e.g., an ED₅₀ for histamine release in a standard in vitro histamine release assay of at least 3 μg/ml, more preferably at least 5 μg/ml, and still more preferably at least 10 μg/ml) and that exhibit water solubility. Histamine-releasing activity may be assayed by, for example, the method described in U.S. Pat. No. 4,851,385 to Roeske. Preferred LHRH antagonists with low histamine-releasing activity and water solubility include compounds disclosed in U.S. Pat. No. 5,843,901 issued Dec. 1, 1998, the entire contents of which are expressly incorporated herein by reference. An especially preferred LHRH antagonist comprises the structure: Ac-D-Nal¹, 4-Cl-D-Phe², D-Pal³, N-Me-Tyr⁵, D-Asn⁶, Lys(iPr)⁸, D-Ala¹⁰-LHRH (referred to herein as abarelix). The efficacy of candidate LHRH antagonists in inhibiting LH release can be assayed, for example, in an animal model such as that described in Corbin and Beattie, Endocrine Res. Commun. 2:1 (1975). In this assay, the LHRH antagonistic activity of a candidate compound is assayed by measuring the antiovulatory activity (AOA) of the compound in rats. The efficacy of candidate LHRH antagonists in inhibiting FSH release can be assayed, for example, using an assay described in Rose et al. Endocrine Reviews 21(1):5-22, the contents of which are incorporated herein by reference.

For reviews of LHRH antagonists, see also B. H. Vickery et al., eds., (1984) “LHRH and Its Analogs: Contraceptive and Therapeutic Applications”, MTP Press Limited, Lancaster, Pa.; and G. Schaison (1989) J. Steroid Biochem. 33(4B): 795. Exemplary LHRH antagonists useful in the methods of the present invention include nonapeptides and decapeptides, as well as peptidomimetics, that mimic the structure of natural LHRH. LHRH antagonists are described in further detail below.

As used herein, the term “selective estrogen receptor modulator” or “SERM” includes a compound, e.g., a drug, which has the ability to modulate the function of an estrogen receptor. Selective estrogen receptor modulators typically act on certain organs as estrogen agonists and on other organs as estrogen antagonists. Selective estrogen receptor modulators can exert known estrogen-like effects on bone and lipids without exerting any action on the endometrium and the breast. Examples of selective estrogen receptor modulators include lasofoxifene, idoxifene, clomifen, tamoxifen, raloxifen (Evista®), droloxifene, levomeloxifene, and toremifine. Selective estrogen receptor modulators are publicly available and described in, for example, Joseph A. Guzzo (2000) Clin. Cardiol. 23, 15-17, U.S. Pat. No. 5,929,090, U.S. Pat. No. 5,962,475, and U.S. Pat. No. 5,811,415, the contents of each of which are incorporated herein by reference.

As used herein, the term “administering” to a subject includes dispensing, delivering or applying an LHRH antagonist and/or a selective estrogen receptor modulator, e.g., an LHRH antagonist and/or a selective estrogen receptor modulator in a pharmaceutical formulation, to a subject by any suitable route for delivery of the composition to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route.

As used herein, the term “effective amount” includes an amount effective, at dosages and for periods of time necessary, to achieve the desired result, e.g., sufficient to treat a hormone associated condition in a subject. An effective amount of an LHRH antagonist and/or a selective estrogen receptor modulator, as defined herein may vary according to factors such as the disease state, age, and weight of the subject, and the ability of the LHRH antagonist and/or the selective estrogen receptor modulator to elicit a desired response in the subject. Dosage regimens may be adjusted to provide the optimum therapeutic response. An effective amount is also one in which any toxic or detrimental effects (e.g., side effects) of the LHRH antagonist and/or the selective estrogen receptor modulator are outweighed by the therapeutically beneficial effects.

In another aspect, the invention features a method for treating vaginal bleeding in a female subject, preferably, a human female. The method includes administering to the subject an LHRH antagonist suitable for in vivo administration and able to reduce both plasma FSH and LH levels in a female subject, in an amount or in a formulation effective to reduce plasma FSH levels in the female subject, e.g., to a symptom alleviating level, thereby treating vaginal bleeding in the female subject.

As used herein, the term “vaginal bleeding” includes bleeding through the vagina of a female subject, other than the normal monthly menstruation based on the menstrual cycle. The term vaginal bleeding includes bleeding of excessive duration or excessive amount; frequent menstruation; intermenstrual bleeding; and postmenopausal bleeding.

In one embodiment, the vaginal bleeding is due to thrombocytopenia, for example, caused by chemotherapy treatment. In another embodiment, the female subject is suffering from a proliferative disorder, e.g., acute myeloid leukemia. In another embodiment, the female subject is a transplant recipient.

As used herein, the term “thrombocytopenia” includes a condition in which the number of blood platelets is decreased, typically resulting in a tendency to bleed from capillaries.

Various aspects of the invention are described further in the following subsections.

LHRH Antagonists

LHRH antagonists preferred for use in the methods of the invention include those described in U.S. Pat. No. 5,843,901, the contents of which are incorporated herein by reference. For example, LHRH antagonists suitable for use in the methods of the invention include peptides comprising a structure:
A-B-C-D-E-F-G-H-I-J,
wherein A is D-Glu, L-Glu, or an analogue thereof; B is D-His, L-His, or an analogue thereof; C is D-Trp, L-Trp, or an analogue thereof; D is D-Ser, L-Ser, or an analogue thereof; E is D-Tyr, L-Tyr, or an analogue thereof; F is D-asparagine, L-asparagine, D-glutamine, or L-glutamine; G is D-Leu, L-Leu or an analogue thereof; H is D-Arg, L-Arg, or an analogue thereof; I is D-Pro, L-Pro, or an analogue thereof; and J is D-Gly, L-Gly, or an analogue thereof; or a pharmaceutically acceptable salt thereof.

In a preferred embodiment, LHRH antagonists suitable for use in the methods of the invention include peptides comprising a structure:
A-B-C-D-E-F-G-H-I,
wherein A is D-Glu, L-Glu, or an analogue thereof; B is D-His, L-His, or an analogue thereof; C is D-Trp, L-Trp, or an analogue thereof; D is D-Ser, L-Ser, or an analogue thereof; E is D-Tyr, L-Tyr, or an analogue thereof; F is D-asparagine, L-asparagine, D-glutamine, or L-glutamine; G is D-Leu, L-Leu or an analogue thereof; H is D-Arg, L-Arg, or an analogue thereof; and I is D-Pro, L-Pro, or an analogue thereof; or a pharmaceutically acceptable salt thereof.

In another embodiment, LHRH antagonists suitable for use in the methods of the invention include peptides comprising a structure:
A-B-C-D-E-F-G-H-I-J
wherein A is pyro-Glu, Ac-Nal, Ac-Qal, Ac-Sar, or Ac-Pal, or an analogue thereof; B is His or 4-Cl-Phe, or an analogue thereof; C is Trp, Pal, Nal, Nal-Pal(N—O), or Trp, or an analogue thereof; D is Ser, or an analogue thereof; E is N-Me-Ala, Tyr, N-Me-Tyr, Ser, Lys(iPr), 4-Cl-Phe, His, Asn, Met, Ala, Arg or Ile, or an analogue thereof; F is Asn or Gln; G is Leu or Trp, or an analogue thereof; H is Lys(iPr), Gln, Met, or Arg, or an analogue thereof; I is Pro, or an analogue thereof; and J is Gly-NH₂ or Ala-NH₂, or an analogue thereof; or a pharmaceutically acceptable salt thereof.

In a preferred embodiment, LHRH antagonists suitable for use in the methods of the invention include peptides comprising a structure:
A-B-C-D-E-F-G-H-I-J
wherein A is pyro-Glu, Ac-Nal, Ac-Qal, Ac-Sar, or Ac-Pal, or an analogue thereof; B is His or 4-Cl-Phe, or an analogue thereof; C is Trp, Pal, Nal, L-Nal-Pal(N—O), or Trp, or an analogue thereof; D is Ser, or an analogue thereof; E is N-Me-Ala, Tyr, N-Me-Tyr, Ser, Lys(iPr), 4-Cl-Phe, His, Asn, Met, Ala, Arg or Ile, or an analogue thereof; F is Asn; G is Leu or Trp, or an analogue thereof; H is Lys(iPr), Gln, Met, or Arg, or an analogue thereof; I is Pro, or an analogue thereof; and J is Gly-NH₂ or Ala-NH₂, or an analogue thereof; or a pharmaceutically acceptable salt thereof.

In one embodiment, LHRH antagonists suitable for use in the methods of the invention include peptides comprising a structure:
A-B-C-D-E-F-G-H-I
wherein A is pyro-Glu, Ac-Nal, Ac-Qal, Ac-Sar, or Ac-Pal, or an analogue thereof; B is His or 4-Cl-Phe, or an analogue thereof; C is Trp, Pal, Nal, Nal-Pal(N—O), or Trp, or an analogue thereof; D is Ser, or an analogue thereof; E is N-Me-Ala, Tyr, N-Me-Tyr, Ser, Lys(iPr), 4-Cl-Phe, His, Asn, Met, Ala, Arg or Ile, or an analogue thereof; F is Asn or Gln; G is Leu or Trp, or an analogue thereof; H is Lys(iPr), Gln, Met, or Arg, or an analogue thereof; and I is Pro, or an analogue thereof; or a pharmaceutically acceptable salt thereof.

In a preferred embodiment, LHRH antagonists suitable for use in the methods of the invention include peptides comprising a structure:
A-B-C-D-E-F-G-H-I
wherein A is pyro-Glu, Ac-Nal, Ac-Qal, Ac-Sar, or Ac-Pal, or an analogue thereof; B is His or 4-Cl-Phe, or an analogue thereof; C is Trp, Pal, Nal, L-Nal-Pal(N—O), or Trp, or an analogue thereof; D is Ser, or an analogue thereof; E is N-Me-Ala, Tyr, N-Me-Tyr, Ser, Lys(iPr), 4-Cl-Phe, His, Asn, Met, Ala, Arg or Ile, or an analogue thereof; F is Asn; G is Leu or Trp, or an analogue thereof; H is Lys(iPr), Gln, Met, or Arg, or an analogue thereof; and I is Pro, or an analogue thereof; or a pharmaceutically acceptable salt thereof.

In another embodiment, LHRH antagonists suitable for use in the methods of the invention include peptides comprising a structure:
A-B-C-D-E-F-G-H-I-J
wherein A is pyro-Glu, Ac-D-Nal, Ac-D-Qal, Ac-Sar, or Ac-D-Pal, or an analogue thereof; B is His or 4-Cl-D-Phe, or an analogue thereof; C is Trp, D-Pal, D-Nal, L-Nal-D -Pal(N—O), or D-Trp, or an analogue thereof; D is Ser, or an analogue thereof; E is N-Me -Ala, Tyr, N-Me-Tyr, Ser, Lys(iPr), 4-Cl-Phe, His, Asn, Met, Ala, Arg or Ile, or an analogue thereof; F is D-Asn or D-Gln; G is Leu or Trp, or an analogue thereof; H is Lys(iPr), Gln, Met, or Arg, or an analogue thereof; I is Pro, or an analogue thereof; and J is Gly-NH₂ or D-Ala-NH₂, or an analogue thereof; or a pharmaceutically acceptable salt thereof.

In a preferred embodiment, LHRH antagonists suitable for use in the methods of the invention include peptides comprising a structure:
A-B-C-D-E-F-G-H-I-J
wherein A is pyro-Glu, Ac-D-Nal, Ac-D-Qal, Ac-Sar, or Ac-D-Pal, or an analogue thereof; B is His or 4-Cl-D-Phe, or an analogue thereof; C is Trp, D-Pal, D-Nal, L-Nal-D -Pal(N—O), or D-Trp, or an analogue thereof; D is Ser, or an analogue thereof; E is N-Me -Ala, Tyr, N-Me-Tyr, Ser, Lys(iPr), 4-Cl-Phe, His, Asn, Met, Ala, Arg or Ile, or an analogue thereof; F is D-Asn; G is Leu or Trp, or an analogue thereof; H is Lys(iPr), Gln, Met, or Arg, or an analogue thereof; I is Pro, or an analogue thereof; and J is Gly-NH₂ or D-Ala-NH₂, or an analogue thereof; or a pharmaceutically acceptable salt thereof.

In another embodiment, LHRH antagonists suitable for use in the methods of the invention include peptides comprising a structure:
A-B-C-D-E-F-G-H-I
wherein A is pyro-Glu, Ac-D-Nal, Ac-D-Qal, Ac-Sar, or Ac-D-Pal, or an analogue thereof; B is His or 4-Cl-D-Phe, or an analogue thereof; C is Trp, D-Pal, D-Nal, L-Nal-D -Pal(N—O), or D-Trp, or an analogue thereof; D is Ser, or an analogue thereof; E is N-Me -Ala, Tyr, N-Me-Tyr, Ser, Lys(iPr), 4-Cl-Phe, His, Asn, Met, Ala, Arg or Ile, or an analogue thereof; F is D-Asn or D-Gln; G is Leu or Trp, or an analogue thereof; H is Lys(iPr), Gln, Met, or Arg, or an analogue thereof; and I is Pro, or an analogue thereof; or a pharmaceutically acceptable salt thereof.

In a preferred embodiment, LHRH antagonists suitable for use in the methods of the invention include peptides comprising a structure:
A-B-C-D-E-F-G-H-I
wherein A is pyro-Glu, Ac-D-Nal, Ac-D-Qal, Ac-Sar, or Ac-D-Pal, or an analogue thereof; B is His or 4-Cl-D-Phe, or an analogue thereof; C is Trp, D-Pal, D-Nal, L-Nal-D -Pal(N—O), or D-Trp, or an analogue thereof; D is Ser, or an analogue thereof; E is N-Me -Ala, Tyr, N-Me-Tyr, Ser, Lys(iPr), 4-Cl-Phe, His, Asn, Met, Ala, Arg or Ile, or an analogue thereof; F is D-Asn; G is Leu or Trp, or an analogue thereof; H is Lys(iPr), Gln, Met, or Arg, or an analogue thereof; and I is Pro, or an analogue thereof; or a pharmaceutically acceptable salt thereof.

In another embodiment, LHRH antagonists suitable for use in the methods of the invention include peptides comprising a structure:
B-C-D-E-F-G-H-I-J,
Wherein B is D-His, L-His, or an analogue thereof; C is D-Trp, L-Trp, or an analogue thereof; D is D-Ser, L-Ser, or an analogue thereof; E is D-Tyr, L-Tyr, or an analogue thereof; F is D-asparagine, L-asparagine, D-glutamine, or L-glutamine; G is D-Leu, L-Leu or an analogue thereof; H is D-Arg, L-Arg, or an analogue thereof; I is D-Pro, L-Pro, or an analogue thereof; and J is D-Gly, L-Gly, or an analogue thereof; or a pharmaceutically acceptable salt thereof.

In a preferred embodiment, LHRH antagonists suitable for use in the methods of the invention include peptides comprising a structure in which the amino acid corresponding to position 6 of the naturally occurring LHRH is D-asparagine or D-glutamine

In another preferred embodiment, LHRH antagonists suitable for use in the methods of the invention include LHRH antagonists which inhibit ovulation in at least 50% of treated rats in a standard rat antiovulatory assay at a dose of 5 μg/rat and which have a low histamine-releasing activity. The term “histamine-releasing activity”, as used herein, refers to the tendency of a compound to release histamine when administered to a subject. The histamine-releasing activity of a compound can be measured with an in vitro assay (described in more detail, infra). Preferred LHRH antagonist peptides have high activity in the rat antiovulatory activity assay, but low histamine releasing activity. Preferred LHRH antagonist peptides have an ED₅₀ in the histamine release assay of at least 3 μg/ml, more preferably at least 5 μg/ml, and still more preferably at least 10 μg/ml.

The LHRH antagonist peptides of the present invention also include peptide analogues. The term “peptide analogue” as used herein is intended to include molecules that mimic the chemical structure of a peptide and retain the functional properties of the peptide. A “residue” includes an amino acid or amino acid analogue incorporated in the peptide compound by an amide bond or amide bond mimetic. The term “amino acid analogue” includes molecules that mimic the chemical structure of naturally-occurring amino acids and that retain the functional properties of naturally-occurring amino acids includes a moiety, other than a naturally occurring amino acid, that conformationally and functionally serves as a substitute for a particular amino acid in a peptide compound without adversely interfering to a significant extent with the function of the peptide (e.g., interaction of the peptide with an LHRH receptor). In some circumstances, substitution with an amino acid analogue may actually enhance properties of the peptide (e.g., interaction of the peptide with an LHRH receptor). Examples of amino acid analogues include D-amino acids. LHRH antagonist peptides substituted with one or more D-amino acids may be made using well known peptide synthesis procedures.

Approaches for designing peptide analogs are known in the art. For example, see Farmer, P. S. in Drug Design (E. J. Ariens, ed.) Academic Press, New York, 1980, vol. 10, pp. 119-143; Ball. J. B. and Alewood, P. F. (1990) J. Mol. Recognition 3:55; Morgan, B. A. and Gainor, J. A. (1989) Ann. Rep. Med. Chem. 24:243; and Freidinger, R. M. (1989) Trends Pharmacol. Sci. 10:270.

Preferred LHRH antagonist peptides suitable for use in the methods of the present invention range in length from about 6 to 15 residues, preferably, from 8 to about 12 residues, more preferably from 9 to 11 residues, and most preferably are 10 residues in length.

The LHRH antagonist peptides of the present invention can be prepared by any suitable method for peptide synthesis, including solution-phase and solid-phase chemical synthesis. Preferably, the peptides are synthesized on a solid support. Methods for chemically synthesizing peptides are well known in the art (see, e.g., Bodansky, M. Principles of Peptide Synthesis, Springer Verlag, Berlin (1993) and Grant, G. A (ed.). Synthetic Peptides: A User's Guide, W.H. Freeman and Company, New York (1992). Automated peptide synthesizers useful to make the peptides of this invention are commercially available.

The use of combinatorial libraries to identify ligands is now well established (see, e.g., M. A. Gallop et al., (1994) J. Med. Chem. 37:1233; and E. M. Gordon et al., (1994) J. Med. Chem. 37:1385; and references cited therein). Therefore, additional LHRH antagonist peptides can be identified by chemical (e.g., solution or solid-phase) synthesis of combinatorial libraries (e.g., of peptides) and screening of the resulting libraries according to known techniques. Thus, many potential ligands can be synthesized and screened in a short period of time, and the most active ligands selected for further testing or use. Using the aforementioned techniques; LHRH antagonists suitable for use in the methods of the present invention may be identified.

As used herein, an LHRH antagonist further includes LHRH antagonists that have been described in the art such as cetrorelix and Nal-Glu; including antagonists described in e.g., U.S. Pat. No. 5,470,947 to Folkers et al.; Folkers et al., PCT Publication No. WO 89/01944; U.S. Pat. No. 5,413,990 to Haviv; U.S. Pat. No. 5,300,492 to Haviv; U.S Pat. No. 5,371,070 to Koerber et al.; U.S. Pat. No. 5,296,468 to Hoeger et al.; U.S. Pat. No. 5,171,835 to Janaky et al.; U.S. Pat. No. 5,003,011 to Coy et al.; U.S. Pat. No. 4,431,635 to Coy; U.S. Pat. No. 4,992,421 to De et al.; U.S. Pat. No. 4,851,385 to Roeske; U.S. Pat. No. 4,801,577 to Nestor, Jr. et al.; and U.S. Pat. No. 4,689,396 to Roeske et al.

Selective Estrogen Receptor Modulators

The methods of the invention include administering to a subject an LHRH antagonist in combination with a selective estrogen receptor modulator (SERM). Any selective estrogen receptor modulator known in the art, such as lasofoxifene, idoxifene, clomifen, tamoxifen, raloxifen (Evista®), droloxifene, levomeloxifene, or toremifine, may be used. Selective estrogen receptor modulators are publicly available and described in, for example, Joseph A. Guzzo (2000) Clin. Cardiol. 23, 15-17, U.S. Pat. No. 5,929,090, U.S. Pat. No. 5,962,475, U.S. Pat. No. 5,811,415, U.S. Pat. No. 4,418,068, U.S. Pat. No. 5,393,763, U.S. Pat. No. 5,843,984, U.S. Pat. No. 5,994,370, U.S. Pat. No. 5,929,090, U.S. Pat. No. 6,153,622, U.S. Pat. No. 5,929,092, U.S. Pat. No. 6,147,092, and U.S. Pat. No. 5,916,916 the contents of each of which are incorporated herein by reference.

Preferred selective estrogen receptor modulators for use in the present invention include modulators that act as agonists in bone tissue and as antagonists in breast and uterine tissue. Preferably, a selective estrogen receptor modulator used in the methods of the invention also acts as an agonist with respect to lipid metabolism.

Pharmaceutical Compositions

LHRH antagonists and/or selective estrogen receptor modulators suitable for use in the methods of the invention can be incorporated into pharmaceutical compositions suitable for administration to a subject, such as those described in U.S. Pat. No. 5,968,895, the contents of which are incorporated herein by reference, which allow for sustained delivery of the LHRH antagonists and/or the selective estrogen receptor modulators for a period of at least several weeks to a month or more. Preferably, an LHRH antagonist and/or a selective estrogen receptor modulator is/are the only active ingredient(s) formulated into the pharmaceutical composition, although in certain embodiments the LHRH antagonist and/or the selective estrogen receptor modulator may be combined with one or more other active ingredients such as an LHRH agonist, or inhibitor of sex steroid biosynthesis. In a preferred embodiment, the pharmaceutical composition comprises an LHRH antagonist, a selective estrogen receptor modulator, and a pharmaceutically acceptable carrier.

As used herein “pharmaceutically acceptable carrier” includes any and all solvents, dispersion media, coatings, antibacterial and antifungal agents, isotonic and absorption delaying agents, and the like that are physiologically compatible. In one embodiment, the carrier is suitable for parenteral administration or for administration via inhalation. Preferably, the carrier is suitable for administration into the central nervous system (e.g., intraspinally or intracerebrally). Alternatively, the carrier can be suitable for intravenous, intraperitoneal or intramuscular administration. In another embodiment, the carrier is suitable for oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, monostearate salts and gelatin. Moreover, the compounds of the invention can be administered in a time release formulation, for example in a composition which includes a slow release polymer. Time release formulations are described in U.S. Pat. No. 5,968,895, incorporated herein in its entirety by reference. The LHRH antagonists and/or the selective estrogen receptor modulators can be prepared with carriers that will protect the LHRH antagonists and/or the selective estrogen receptor modulators against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the LHRH antagonists and/or the selective estrogen receptor modulators in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The LHRH antagonists and/or the selective estrogen receptor modulators can be formulated with one or more additional compounds that enhance the solubility of the LHRH antagonists and/or the selective estrogen receptor modulators. Preferred compounds to be added to formulations to enhance the solubility of the LHRH antagonists and/or the selective estrogen receptor modulators are cyclodextrin derivatives, preferably hydroxypropyl-γ-cyclodextrin. For example, inclusion in the formulation of hydroxypropyl-γ-cyclodextrin at a concentration 50-200 mM may increase the aqueous solubility of the LHRH antagonists and/or the selective estrogen receptor modulators.

Another formulation for the LHRH antagonists and/or the selective estrogen receptor modulators comprises the detergent Tween-80, polyethylene glycol (PEG) and ethanol in a saline solution. A non-limiting example of such a preferred formulation is 0.16% Tween-80, 1.3% PEG-3000 and 2% ethanol in saline.

Preferably, the LHRH antagonist is administered to the subject as a sustained-release formulation using a pharmaceutical composition comprising a solid ionic complex of an LHRH antagonist and a carrier macromolecule, wherein the carrier and LHRH antagonist used to form the complex are combined at a weight ratio of carrier:LHRH antagonist of for example, 0.5:1 to 0.1:1. In other embodiments, the carrier and LHRH antagonist used to form the complex are combined at a weight ratio of carrier:LHRH antagonist of 0.8:1, 0.7:1, 0.6:1, 0.5:1, 0.4:1, 0.3:1, 0.25:1, 0.2:1, 0.15:1, or 0.1:1. In a preferred embodiment, the complex is not a microcapsule. Ranges intermediate to the above recited values, e.g., 0.8:1 to 0.4:1, 0.6:1 to 0.2:1, or 0.5:1 to 0.1:1 are also intended to be part of this invention. For example, ranges of values using a combination of any of the above recited values as upper and/or lower limits are intended to be included.

In another embodiment, the LHRH antagonist is administered to the subject using a pharmaceutical composition comprising a solid ionic complex of an LHRH antagonist and a carrier macromolecule, wherein the LHRH antagonist content of said complex is at least 40% by weight, preferably at least 45%, 50%, 55%, 57%, 60%, 65%, 70%, 75%, 80%, 85%, 90%, or 95% by weight. Ranges intermediate to the above recited values, e.g., at least about 50% to about 80%, at least about 60% to about 90%, or at least about 57% to about 80%, are also intended to be part of this invention. For example, ranges of values using a combination of any of the above recited values as upper and/or lower limits are intended to be included.

As used herein, the term “carrier macromolecule” is intended to refer to a macromolecule that can complex with a peptide to form a water-insoluble complex. Preferably, the macromolecule has a molecular weight of at least 5 kDa, more preferably at least 10 kDa. The term “anionic carrier macromolecule” is intended to include negatively charged high molecular weight molecules, such as anionic polymers. The term “cationic carrier macromolecule” is intended to include positively charged high molecular weight molecules, such as cationic polymers.

As used herein, the term “water-insoluble complex” is intended to refer to a physically and chemically stable complex that forms upon appropriate combining of an LHRH antagonist and carrier macromolecule according to procedures described herein. This complex typically takes the form of a precipitate that is produced upon combining aqueous preparations of the LHRH antagonist and carrier macromolecule. Although not intending to be limited by mechanism, the formation of preferred water-insoluble complexes used in the methods of the invention is thought to involve (e.g., be mediated at least in part by) ionic interactions in situations where the LHRH antagonist is cationic and the carrier molecule is anionic or vice versa. Additionally or alternatively, the formation of a water-insoluble complex of the invention may involve (e.g., be mediated at least in part by) hydrophobic interactions. Still further, formation of a water-insoluble complex of the invention may involve (e.g., be mediated at least in part by) covalent interactions. Description of the complex as being “water-insoluble” is intended to indicate that the complex does not substantially or readily dissolve in water, as indicated by its precipitation from aqueous solution. However, it should be understood that a “water-insoluble” complex of the invention may exhibit limited solubility in water either in vitro or in the aqueous physiological environment in vivo.

As used herein, the term “sustained delivery” or “sustained release” is intended to refer to continual delivery of an LHRH antagonist and/or a selective estrogen receptor modulator in vivo over a period of time following administration, preferably at least several days, a week or several weeks and up to a month or more. In a preferred embodiment, a formulation of the invention achieves sustained delivery for at least about 28 days, at which point the sustained release formulation can be re-administered to achieve sustained delivery for another 28 day period (which re-administration can be repeated every 28 days to achieve sustained delivery for several months to years). Sustained delivery of the LHRH antagonist and/or the selective estrogen receptor modulator can be demonstrated by, for example, the continued therapeutic effect of the LHRH antagonist and/or the selective estrogen receptor modulator over time. Alternatively, sustained delivery of the LHRH antagonist and/or the selective estrogen receptor modulator may be demonstrated by detecting the presence of the LHRH antagonist and/or the selective estrogen receptor modulator in vivo over time.

A complex used in the methods of the invention is prepared by combining the LHRH antagonist and the carrier macromolecule under conditions such that a water-insoluble complex of the LHRH antagonist and the carrier macromolecule forms.

For example, a solution of the LHRH antagonist and a solution of the carrier macromolecule are combined until a water-insoluble complex of the LHRH antagonist and the carrier macromolecule precipitates out of solution. In certain embodiments, the solutions of the LHRH antagonist and the carrier macromolecule are aqueous solutions.

Alternatively, if the LHRH antagonist or the carrier molecule (or both) is not substantially water soluble prior to combination the two, then the LHRH antagonist and/or carrier macromolecule can be dissolved in a water-miscible solvent, such as an alcohol (e.g., ethanol) prior to combining the two components of the complex. In another embodiment of the method of preparing the water-insoluble complex, the solution of the LHRH antagonist and the solution of the carrier macromolecule are combined and heated until a water-insoluble complex of the LHRH antagonist and the carrier macromolecule precipitates out of solution. The amounts of LHRH antagonist and carrier macromolecule necessary to achieve the water-insoluble complex may vary depending upon the particular LHRH antagonist and carrier macromolecule used, the particular solvent(s) used and/or the procedure used to achieve the complex. Typically, however, the LHRH antagonist will be in excess relative to the anionic molecule on a molar basis. Often, the LHRH antagonist also will be in excess on a weight/weight basis, as indicated above. In certain embodiments, the carrier macromolecule is preferably carboxymethylcellulose, and the LHRH antagonist is preferably abarelix.

Once the LHRH antagonist/macromolecule complex precipitates out of solution, the precipitate can be removed from the solution by means known in the art, such as filtration (e.g., through a 0.45 micron nylon membrane), centrifugation and the like. The recovered paste then can be dried (e.g., in vacuum or in a 70° C. oven) and the solid can be milled or pulverized to a powder by means known in the art (e.g., hammer or gore milling, or grinding in mortar and pestle). Alternatively, the paste can be frozen and lyophilized to dryness. The powder form of the complex can be dispersed in a carrier solution to form a liquid suspension or semi-solid dispersion suitable for injection.

Accordingly, in various embodiments, a pharmaceutical formulation of the invention is a lyophilized solid, a liquid suspension or a semi-solid dispersion.

In another embodiment, the pharmaceutical formulation used in the methods of the invention is a sterile formulation. For example, following formation of the water-insoluble complex, the complex can be sterilized, preferably by gamma irradiation or electron beam sterilization. Alternatively, to prepare a sterile pharmaceutical formulation, the water-insoluble complex can be isolated using conventional sterile techniques (e.g., using sterile starting materials and carrying out the production process aseptically).

The pharmaceutical formulation can be administered to the subject by any route suitable for achieving the desired therapeutic result(s), although preferred routes of administration are parenteral routes, in particular intramuscular (i.m.) injection and subcutaneous/intradernal (s.c./i.d.) injection. Alternatively, the formulation can be administered to the subject orally. Other suitable parental routes include intravenous injection, buccal administration, transdermal delivery and administration by the rectal, vaginal, intranasal or respiratory tract route. It should be noted that when a formulation that provides sustained delivery for weeks to months by the i.m or s.c./i.d. route is administered by an alternative route, there may not be sustained delivery of the agent for an equivalent length of time due to clearance of the agent by other physiological mechanisms (i.e., the dosage form may be cleared from the site of delivery such that prolonged therapeutic effects are not observed for time periods as long as those observed with i.m or s.c./i.d. injection).

The pharmaceutical formulation contains a therapeutically effective amount of the LHRH antagonist and/or the selective estrogen receptor modulator. A “therapeutically effective amount” refers to an amount effective, at dosages and for periods of time necessary, to achieve the desired result. A therapeutically effective amount of an LHRH antagonist and/or a selective estrogen receptor modulator may vary according to factors such as the disease state, age, and weight of the individual, and the ability of the LHRH antagonist and/or the selective estrogen receptor modulator (alone or in combination with one or more other drugs) to elicit a desired response in the subject. Dosage regimens may be adjusted to provide the optimum therapeutic response. A therapeutically effective amount is also one in which any toxic or detrimental effects of the LHRH antagonist and/or the selective estrogen receptor modulator are outweighed by the therapeutically beneficial effects.

In one embodiment, the dosage of the LHRH antagonist and/or the selective estrogen receptor modulator is about 10-500 mg/month, about 20-300 mg/month, or about 30-200 mg/month. In a preferred embodiment, the dosage of the LHRH antagonist and/or the selective estrogen receptor modulator is about 30-120 mg/month. Ranges intermediate to the above recited values, e.g., about 10-200 mg/month, about 30-250mg/month, or about 100-200 mg/month, are also intended to be part of this invention. For example, ranges of values using a combination of any of the above recited values as upper and/or lower limits are intended to be included. The above recited dosages may also be calculated and expressed in mg/kg/day. Accordingly, in another embodiment, the dosage of the LHRH antagonist and/or the selective estrogen receptor modulator is about 5-500 μg/kg/day, about 10-400 μg/kg/day, or about 20-200 μg/kg/day. In a preferred embodiment, the dosage of the LHRH antagonist and/or the selective estrogen receptor modulator is about 100 μg/kg/day. It is to be noted that dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Methods for Treating a Hormone Associated Condition in a Subject

In another embodiment, the present invention provides a method for treating (e.g., therapeutically or prophylactically) a hormone associated condition in a subject. The method includes administering to a subject a combination of an LHRH antagonist and a selective estrogen receptor modulator, thereby treating a hormone associated condition in the subject.

As used herein, the term “hormone associated condition” includes any disease, disorder, or condition associated with a sex hormone, e.g., estrogen or progesterone. Hormone associated conditions include conditions, diseases, or disorders which affect the organs of the reproductive system, e.g., the uterus or the vagina; conditions, diseases, or disorders which involve an imbalance in the levels of a reproductive-hormone in a subject; and conditions, diseases, or disorders affecting the ability of a subject to reproduce. Examples of hormone associated conditions include endometriosis, vaginal bleeding, infertility, ovarian cancer, uterine fibroids, breast cancer, premenstrual syndrome, polycystic ovary syndrome, ovarian cysts, uterine leiomata, and dysfunctional uterine bleeding.

As used herein, the term “subject” includes warm-blooded animals, preferably mammals, including humans. In a preferred embodiment, the subject is a primate. In an even more preferred embodiment, the primate is a human.

As used herein, the term “administering” to a subject includes dispensing, delivering or applying an LHRH antagonist and/or a selective estrogen receptor modulator, e.g., an LHRH antagonist and/or a selective estrogen receptor modulator in a pharmaceutical formulation (as described herein), to a subject by any suitable route for delivery of the compound to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route.

As used herein, the term “effective amount” includes an amount effective, at dosages and for periods of time necessary, to achieve the desired result, e.g., sufficient to treat a hormone associated disorder in a subject. An effective amount of an LHRH antagonist and/or a selective estrogen receptor modulator, as defined herein may vary according to factors such as the disease state, age, and weight of the subject, and the ability of the LHRH antagonist and/or the selective estrogen receptor modulator to elicit a desired response in the subject. Dosage regimens may be adjusted to provide the optimum therapeutic response. An effective amount is also one in which any toxic or detrimental effects (e.g., side effects) of the LHRH antagonist and/or the selective estrogen receptor modulator are outweighed by the therapeutically beneficial effects.

A therapeutically effective amount of an LHRH antagonist and/or a selective estrogen receptor modulator (i.e., an effective dosage) may range from about 0.001 to 80 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an LHRH antagonist and/or a selective estrogen receptor modulator can include a single treatment or, preferably, can include a series of treatments. In one example, a subject is treated with an LHRH antagonist and/or a selective estrogen receptor modulator in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of an LHRH antagonist and/or a selective estrogen receptor modulator used for treatment may increase or decrease over the course of a particular treatment.

The methods of the invention further include administering to a subject a therapeutically effective amount of an LHRH antagonist and/or a selective estrogen receptor modulator in combination with another pharmaceutically active compound known to treat a hormone associated disorder. Other pharmaceutically active compounds that may be used can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell New Jersey, Medical Economics Co., the complete contents of which are expressly incorporated herein by reference. The LHRH antagonist and the selective estrogen receptor modulator may be administered to the subject in the same pharmaceutical composition or in different pharmaceutical compositions (at the same time or at different times). The LHRH antagonist and/or the selective estrogen receptor modulator and the additional pharmaceutically active compound may be administered to the subject in the same pharmaceutical composition or in different pharmaceutical compositions (at the same time or at different times).

In one aspect, the invention provides a method for preventing in a subject, a hormone associated condition, by administering to the subject a combination of an LHRH antagonist and a selective estrogen receptor modulator. Administration of a combination of an LHRH antagonist and a selective estrogen receptor modulator can occur prior to the manifestation of symptoms characteristic of the hormone associated condition, such that such a condition, e.g., breast or ovarian cancer, is prevented or, alternatively, delayed in its progression.

The contents of all references, patents and published patent applications cited throughout this application, as well as the Figures, are hereby incorporated by reference.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method for treating a hormone associated condition in a subject, comprising administering to a subject a combination of an LHRH antagonist and a selective estrogen receptor modulator, thereby treating a hormone associated condition in the subject.

2. The method of claim 1, wherein the hormone associated condition is endometriosis.

3. The method of claim 1, wherein the hormone associated condition is ovarian cancer.

4. The method of claim 1, wherein the hormone associated condition is breast cancer.

5. The method of claim 1, wherein the hormone associated condition is polycystic ovary syndrome.

6. The method of claim 1, wherein the hormone associated condition is uterine leiomata.

7. The method of claim 1, wherein the hormone associated condition is dysfunctional uterine bleeding.

8. The method of claim 1, wherein the hormone associated condition is premenstrual syndrome.

9. The method of claim 1, wherein the hormone associated condition is vaginal bleeding.

10. The method of claim 1, wherein the hormone associated condition is uterine fibroids.

11. The method of claim 1, wherein the subject is a mammal.

12. The method of claim 1, wherein the subject is a human.

13. The method of claim 1, wherein the LHRH antagonist has an ED₅₀ for histamine release in a standard in vitro histamine release assay of at least 3 μg/ml.

14. The method of claim 1, wherein the LHRH antagonist has an ED₅₀ for histamine release in a standard in vitro histamine release assay of at least 5 μg/ml.

15. The method of claim 1, wherein the LHRH antagonist has an ED₅₀ for histamine release in a standard in vitro histamine release assay of at least 10 μg/ml.

16. The method of claim 1, wherein the LHRH antagonist is a decapeptide or a nonapeptide compound having a D-asparagine, an L-asparagine, a D-glutamine, or an L-glutamine at a position corresponding to position 6 of naturally occurring LHRH, or a pharmaceutically acceptable salt thereof.

17. The method of claim 16, wherein the LHRH antagonist is a decapeptide.

18. The method of claim 16, wherein the LHRH antagonist is a nonapeptide.

19. The method of claim 1, wherein the LHRH antagonist is a peptide compound comprising a structure:
A-B-C-D-E-F-G-H-I-J
wherein
A is pyro-Glu, Ac-D-Nal, Ac-D-Qal, Ac-Sar, or Ac-D-Pal, or an analogue thereof;
B is His or 4-Cl-D-Phe, or an analogue thereof;
C is Trp, D-Pal, D-Nal, L-Nal- D-Pal(N—O), or D-Trp, or an analogue thereof;
D is Ser, or an analogue thereof;
E is N-Me-Ala, Tyr, N-Me-Tyr, Ser, Lys(iPr), 4-Cl-Phe, His, Asn, Met, Ala, Arg or Ile, or an analogue thereof;
F is D-Asn or D-Gln;
G is Leu or Trp, or an analogue thereof;
H is Lys(iPr), Gln, Met, or Arg, or an analogue thereof;
I is Pro, or an analogue thereof; and
J is Gly-NH₂ or D-Ala-NH₂, or an analogue thereof;
or a pharmaceutically acceptable salt thereof.

20. The method of claim 1, wherein the LHRH antagonist is a peptide compound comprising a structure:
A-B-C-D-E-F-G-H-I-J
wherein
A is pyro-Glu, Ac-D-Nal, Ac-D-Qal, Ac-Sar, or Ac-D-Pal, or an analogue thereof;
B is His or 4-Cl-D-Phe, or an analogue thereof;
C is Trp, D-Pal, D-Nal, L-Nal- D-Pal(N—O), or Trp, or an analogue thereof;
D is Ser, or an analogue thereof;
E is N-Me-Ala, Tyr, N-Me-Tyr, Ser, Lys(iPr), 4-Cl-Phe, His, Asn, Met, Ala, Arg or Ile, or an analogue thereof;
F is D-Asn;
G is Leu or Trp, or an analogue thereof;
H is Lys(iPr), Gln, Met, or Arg, or an analogue thereof;
I is Pro, or an analogue thereof; and
J is Gly-NH₂ or D-Ala-NH₂, or an analogue thereof;
or a pharmaceutically acceptable salt thereof.

21. The method of claim 1, wherein the LHRH antagonist is a peptide compound comprising a structure:
Ac-D-Nal-4-Cl-D-Phe-D-Pal-Ser-N-Me-Tyr-D-Asn-Leu-Lys(iPr)-Pro-D-Ala-NH₂;
or a pharmaceutically acceptable salt thereof.

22. The method of claim 1, wherein the LHRH antagonist is a peptide compound comprising a structure:
Ac-D-Nal-4-Cl-D-Phe-D-Pal-Ser-Tyr-D-Asn-Leu-Lys(iPr)-Pro-D-Ala-NH₂;
or a pharmaceutically acceptable salt thereof.

23. The method of claim 1, wherein the selective estrogen receptor modulator is raloxifene.

24. The method of claim 1, wherein the selective estrogen receptor modulator is tamoxifen.

25. The method of claim 1, wherein the LHRH antagonist and the selective estrogen receptor modulator are administered to the subject using a sustained-release formulation.

26. The method of claim 25, wherein the sustained-release formulation of LHRH antagonist comprises a solid ionic complex of an LHRH antagonist and a carrier macromolecule, wherein the carrier and LHRH antagonist used to form the complex are combined at a weight ratio of carrier:antagonist of 0.5:1 to 0.1:1.

27. The method of claim 1, wherein the LHRH antagonist and the selective estrogen receptor modulator are administered at a dosage of about 5-500 μg/kg/day.

28. The method of claim 1, wherein the LHRH antagonist and the selective estrogen receptor modulator are administered at a dosage of about 10-400 μg/kg/day.

29. The method of claim 1, wherein the LHRH antagonist and the selective estrogen receptor modulator are administered at a dosage of about 10-100 μg/kg/day.

30. The method of claim 1, wherein the LHRH antagonist and the selective estrogen receptor modulator are administered to the subject simultaneously.

31. The method of claim 1, wherein the LHRH antagonist and the selective estrogen receptor modulator are administered to the subject at different times.

32. The method of claim 1, wherein the LHRH antagonist and the selective estrogen receptor modulator are administered to the subject in the same formulation.

33. The method of claim 1, wherein the LHRH antagonist and the selective estrogen receptor modulator are administered to the subject in separate formulations.

34. A method for treating endometriosis in a subject, comprising administering to a subject a combination of an LHRH antagonist and a selective estrogen receptor modulator, thereby treating endometriosis in the subject.

35. A method for treating ovarian cancer in a subject, comprising administering to a subject a combination of an LHRH antagonist and a selective estrogen receptor modulator, thereby treating ovarian cancer in the subject.

36. A method for treating breast cancer in a subject, comprising administering to a subject a combination of an LHRH antagonist and a selective estrogen receptor modulator, thereby treating breast cancer in the subject.

37. A method for treating polycystic ovary syndrome in a subject, comprising administering to a subject a combination of an LHRH antagonist and a selective estrogen receptor modulator, thereby treating polycystic ovary syndrome in the subject.

38. A method for treating uterine leiomata in a subject, comprising administering to a subject a combination of an LHRH antagonist and a selective estrogen receptor modulator, thereby treating uterine leiomata in the subject.

39. A method for treating dysfunctional uterine bleeding in a subject, comprising administering to a subject a combination of an LHRH antagonist and a selective estrogen receptor modulator, thereby treating dysfunctional uterine bleeding in the subject.

40. A method for treating premenstrual syndrome in a subject, comprising administering to a subject a combination of an LHRH antagonist and a selective estrogen receptor modulator, thereby treating premenstrual syndrome in the subject.

41. A method for treating vaginal bleeding in a subject, comprising administering to a subject a combination of an LHRH antagonist and a selective estrogen receptor modulator, thereby treating vaginal bleeding in the subject.

42. The method of claim 41, wherein the vaginal bleeding is due to thrombocytopenia.

43. The method of claim 42, wherein the thrombocytopenia is caused by chemotherapy treatment.

44. The method of claim 41, wherein the subject is suffering from a proliferative disorder.

45. The method of claim 44, wherein the proliferative disorder is acute myeloid leukemia.

46. A method for treating uterine fibroids in a subject, comprising administering to a subject a combination of an LHRH antagonist and a selective estrogen receptor modulator, thereby treating uterine fibroids in the subject.
